(19) United States
(12) Patent Application Publication
(10) Pub. No.: US 2003/0204417 A1
(43) Pub. Date: Oct. 30, 2003

(54) Apparatus, method and system for the recordation, retrieval and management of pet information and capitalization of same

(76) Inventor: John Hannis Mize, Alpharetta, GA (US)

Correspondence Address: MYERS & KAPLAN, INTELLECTUAL, PROPERTY LAW, L.L.C., 1827 POWERS FERRY ROAD, BUILDING 3, SUITE 200,, ATLANTA, GA, 30339, US

(21) Appl. No.: 10/439,605

(22) Filed: May 16, 2003

(51) Int. Cl. G06F 17/60; A01K 29/00; A61B 5/00

(52) U.S. Cl. 705/2; 119/174; 235/385; 600/300; 705/14

(57) ABSTRACT

An apparatus, method and system for the recordation, retrieval and management of pet information and capitalization of same, wherein the present invention provides for the recordation and membership-based selective access and retrieval of pet or animal medical conditions, drug allergies, vaccination history and other relevant health or identification related information, and wherein the present invention further provides for the recordation and sponsorship-based selective access and retrieval of pet or animal owner spending habits and expenses attributed to the general maintenance and care of the pet or animal, thus permitting animal food and drug providers, veterinary hospitals, insurance carriers and/or other pet related product or service providers to conduct market analyses of consumer demographics, demands, needs and spending habits for subsequent capitalization upon same via direct marketing, promotional discount offers and the like.

### TECHNICAL FIELD

The present invention relates generally to information storage and retrieval devices and systems, and more specifically to an apparatus, method and system for the recordation, retrieval and management of pet information and capitalization of same, wherein the present invention is particularly suitable for, although not strictly limited to, the recordation and selective access or retrieval of pet medical history and/or examinations, and pet owner spending habits or expenses attributed to the general maintenance and care of the pet.

### BACKGROUND OF THE INVENTION

Modern advancements in veterinary medicine have promoted healthier, longer lives for domesticated and livestock animals alike. Specifically, with regular advents in preventative medicine and nutritional and dietary sciences, veterinarians are able to more prudently implement current medical treatments and establish nutritional or dietary regimens that best suit or compliment a particular breed or species of animal, thereby facilitating a healthier lifestyle and increasing overall lifespan of same.

In addition to veterinary efforts to promote healthier, longer lives for pets, or animals in general, pet owners and/or animal breeders also often dedicate a substantial amount of time and energy to the healthy maintenance of their pets or animals, wherein such maintenance includes, but is not limited to, regular veterinary visits, proper in-home administration of medical treatment and/or the establishment and governance of appropriate eating or dietary habits.

However, an obvious result of such increased animal health management and treatment is the production and over accumulation of medical or veterinary records and related documentation, as well as animal products, for each pet or animal. Unfortunately, such increases in records and documentation often delay attempts by the pet owner or veterinarian to expeditiously and conveniently retrieve information regarding an animal's existing medical condition, drug allergies, vaccination history, or other basic animal health or identification related information.

Additionally, as animal breeders (and animal handlers and humane societies) often concurrently manage a multitude of animals, and veterinarians a multitude of animal patients, animal breeders, veterinarians and/or other animal care-providers are often forced to search through extensive animal medical records and documentation for retrieval of otherwise simple information for a specific animal or pet. Such lengthy delays may present fatal consequences in circumstances where the animal or pet requires immediate medical attention, but is deprived of such attention due to the inability of the care-provider to expeditiously retrieve drug allergy information or existing medical conditions for proper administration of first response medical action.

Furthermore, many pet owners take their pets with them while traveling, vacationing or departing from their residence for any length of time. However, most such pet owners do not generally transport with them their pet's medical or health records, as such a task would be characteristically burdensome and is generally not considered a necessary item for travel. Unfortunately, injuries, accidents, poisonings or other medical emergencies are rarely foreseeable, and without immediate access to a pet's basic medical information, proper medical attention by the nearest veterinary hospital may be delayed until information regarding drug allergies, existing medical conditions, or the medical records themselves are obtained. Furthermore, those pets at most risk of suffering from such delayed medical attention are lost or runaway pets that have been found by non-owners and admitted to a local shelter or veterinary hospital for medical treatment, wherein such local shelters or veterinary hospitals are not likely to possess the animal's or pet's medical records.

Increased animal health management and treatment has also affected the animal food and drug industries. More specifically, with advancements in veterinary sciences and a more demanding consumer market (i.e., pet or animal owners or breeders), animal food and drug manufacturers, distributors and retailers (i.e., animal food and drug providers, in general) are striving to meet the ever-increasing veterinary and consumer demands for high-quality food and drug products. However, without full knowledge of specific consumer demographics, demands and spending habits, animal food and drug providers are often unable to supply or meet the specific demands of a specific consumer populace, and thus, often supply such consumers with inadequate amounts of a desired product, an over abundance of an undesired product, or an unappealing or unimpressive standard inventory. Moreover, although such products may be of high-quality and rightfully accorded name-brand status, some buyers may simply be reluctant to spend the extra money in purchasing the product absent a promotional discount, or the like.

However, without the ability of animal food and drug providers to directly target, and thus maintain, discount-driven or thrifty buyers via direct promotional offers or discounts, such providers must often cut into their profit margin to provide discounted product sales to the general public in attempts to recapture the business of such thrifty buyers. Unfortunately, however, animal food and drug providers currently lack any convenient, systematic method of recording, retrieving and managing consumer demographics, demands and spending habits for improving product marketing thereto, whether via direct consumer marketing or promotional discount offers.

Animal health insurance industries have benefited from increased animal health management and treatment, as veterinary medical procedures, treatments, examinations and drug prescriptions are often too expensive to be routinely paid in full by the pet or animal owner. However, many pet or animal owners are typically unapprised of the benefits, types of policies and scope of coverage offered by pet or animal health insurance plans, and, as such, often avoid pursing and implementing same. Although insurance companies possess indirect methods of advertising and promotional offerings (i.e., via a global communications network, television and radio commercials, or written communications provided at a local veterinarian hospital or animal shelter), targeted communications to a specific group of consumers (i.e., those with pets or animal) would facilitate a more effective means of services advertising and promotional offerings. However, insurance companies currently lack any convenient, systematic method of recordation, retrieval and management of consumer demographics and insurance needs.

Therefore, it is readily apparent that there is a need for an apparatus, method and system for the recordation, retrieval and management of pet information and capitalization of same, wherein the present invention provides for the recordation and selective retrieval of pet or animal medical, health or identification related information, and wherein the present invention further supplies animal food and drug providers, insurance carriers and/or pet related product or service vendors with a systematic method of recordation, retrieval and management of consumer demographics, demands, needs and spending habits for improving product and/or service marketing thereto, and for capitalizing upon same.

### BRIEF SUMMARY OF THE INVENTION

Briefly described, in a preferred embodiment, the present invention overcomes the above-mentioned disadvantages and meets the recognized need for such a device by providing an apparatus, method and system for the recordation, retrieval and management of pet information and capitalization of same, wherein the present invention provides for the recordation and membership-based selective access and retrieval of pet or animal medical conditions, drug allergies, vaccination history and other relevant health or identification related information, and wherein the present invention further provides for the recordation and sponsorship-based selective access and retrieval of pet or animal owner spending habits and expenses attributed to the general maintenance and care of the pet or animal, thus permitting animal food and drug providers, insurance carriers and/or other pet related product or service providers to conduct market analyses of consumer demographics, demands, needs and spending habits for subsequent capitalization upon same via direct marketing, promotional discount offers and the like.

According to its major aspects and broadly stated, the present invention in its preferred form is an apparatus, method and system for the recordation, retrieval and management of pet information and capitalization of same, having an information or identification card for in-hand and/or remote recordation, membership-based selective retrieval and management of pertinent pet or animal information, and a hierarchically-based sponsorship program that governs a particular sponsor's type and depth of access and/or retrieval of pet or animal owner spending habits and expenses attributed to the general maintenance and care of the pet or animal, thus permitting the sponsor to conduct market analyses of consumer demographics, demands, needs and spending habits for subsequent capitalization upon same via direct consumer marketing, promotional discount offers and the like.

More specifically, the present invention is an apparatus, method and system for the recordation, retrieval and management of pet information and capitalization of same, having a vendor-supplied information or identification card for consumer (i.e., cardholder) in-hand and/or remote recordation, retrieval and management of pet or animal medical conditions, drug allergies, vaccination history and other relevant health and identification related information, wherein a membership-based program preferably governs a particular member's (i.e., cardholder's) type and depth of access to such information. The information is preferably utilized by the pet owner (i.e., member or cardholder) and/or veterinarian for implementation of appropriate animal care, medical procedures, examinations and treatments. Although the card preferably possesses written information or pet identifying indicia thereon, additional information may be recorded, retrieved, stored and managed thereon via a variety of input/output recordation, storage and retrieval means, such as, for exemplary purposes only, bar code technology, magnetic strips, and/or microchip technology.

Furthermore, all such relevant pet health and identification related information is preferably conveyed to the original card vendor (i.e., preferably operating under the trademarks PPI and/or 4PETSID) for establishing a database of information for each cardholder (i.e., member). As such, should a cardholder's pet or animal become lost, the original card vendor can, via a vendor-established (i.e., PPI-established or 4PETSID-established) database of veterinary hospitals, animal shelters and/or pet stores, effectuate a mass mailing, facsimile, or the like, that would contain pet health and identification related information for use by the finder of the lost pet, thus facilitating medical treatment of same and a more promising and expeditious reunion between the pet and pet owner.

The card further provides a variety of promotional offers and discounts on certain products and services, thereby encouraging cardholders to present their cards upon purchasing pet food and drug products, paying for veterinarian services, or the like. Each time the card is presented, it may be scanned (i.e., via the aforementioned bar code technology, magnetic strips, microchip technology, or the like) and therefore record and track, the purchased products or services. As such, the card records and tracks consumer spending habits and expenses attributed to the general maintenance and care of the pet or animal, wherein such information is then conveyed to the original card vendor for recordation and subsequent distribution via sponsorship programs to animal food and drug providers, insurance carriers and/or other pet related product or service providers, as more fully detailed below. With such information, the original card vendor, or others, are able to analyze consumer spending habits for determination of consumer geographic location, species of pet, desirable or undesirable product lines, profitable retail locations, frequency of purchases, typical fees associated with veterinary services, effective food and drug products, or the like, wherein such market determinations and analyses are selectively accessible by sponsors of the present inventive system and method for subsequent capitalization upon same via direct consumer marketing, promotional discount offers, or the like, as described below.

The present invention further provides a method and system for the retrieval and management of such consumer spending habit information, wherein a hierarchically-based sponsorship program preferably governs a particular sponsor's type and depth of access and/or retrieval of pet or animal owner spending habits and expenses attributed to the general maintenance and care of the pet or animal, thus permitting the sponsor to conduct market analyses of consumer demographics, demands, needs and spending habits for subsequent capitalization upon same via direct marketing, promotional discount offers, or the like.

Specifically, sponsor logos are placed upon the card upon payment by the sponsor of a minimum sponsorship fee to the original card vendor, wherein the minimum sponsorship fee functions to convey only basic advertising rights to the sponsor by way of the logo on the card. Should the sponsor desire to access and retrieve all the aforementioned consumer spending habit information, the sponsor pays an additional fee to the original card vendor. Additionally, the sponsor may elect to pay the original card vendor another fee that would entitle the sponsor to access mailing, emailing or other similar contact information of a specific consumer or cardholder for the direct marketing mailing, promotional and discount offerings thereto.

Accordingly, a feature and advantage of the present invention is its ability to provide an apparatus, method and system for the recordation, retrieval and management of pet information and capitalization of same.

Another feature and advantage of the present invention is its ability to provide for the recordation and membership-based selective retrieval and management of pet or animal medical conditions, drug allergies, vaccination history and other relevant pet health and identification related information, wherein a membership-based program preferably governs a particular member's (i.e., cardholder's) type and depth of access to such information.

Another feature and advantage of the present invention is its hierarchically-based sponsorship program that governs a particular sponsor's type and depth of access and/or retrieval of pet or animal owner spending habits and expenses attributed to the general maintenance and care of the pet or animal.

Another feature and advantage of the present invention is its ability to permit sponsors to conduct market analyses of consumer demographics, demands, needs and spending habits for subsequent capitalization upon same via direct consumer marketing, promotional discount offers and the like.

Another feature and advantage of the present invention is its ability to permit in-hand and/or remote recordation, retrieval and management of pet or animal owner spending habits and expenses attributed to the general maintenance and care of the pet or animal for subsequent access, management and capitalization upon same by product or service sponsors, pet stores, veterinarians, or the like.

Another feature and advantage of the present invention is its ability to facilitate the locating and medical treatment of lost pets, and the expeditious reunion between the pet and pet owner.

Another feature and advantage of the present invention is its ability to promote higher quality health care for animals by providing pet or animal owners with the ability to increase pet or animal veterinarian visits and product/service purchases at discounted rates, thereby reducing high costs and expenses typically associated with same.

Another feature and advantage of the present invention is its ability to provide a variety of promotional offers and discounts on certain products and services, thereby encouraging cardholders to present their cards upon purchasing products and services, and thus, the recording and tracking of same.

Another feature and advantage of the present invention is its ability to record and provide information to the original card vendor, or others, for the determination of consumer spending habits, consumer geographic location, species of pet, desirable or undesirable product lines, profitable retail locations, frequency of purchases, typical fees associated with veterinary services, effective food and drug products, or the like.

Another feature and advantage of the present invention is its ability to provide a means for advertising and marketing for the original card vendor, veterinarian or animal hospital, animal food and drug providers, insurance carriers and/or other pet related product or service providers.

Another feature and advantage of the present invention is its ability to streamline inventory for veterinarians, or the like, and promote goodwill for hospitals carrying discounted products manufactured by sponsor's of card.

Another feature and advantage of the present invention is its ability to increase the bottom line of veterinarians, pet stores, or the like, carrying member incentive discounted products by significantly reducing unnecessary expenditures and inventory.

These and other objects, features and advantages of the present invention will become more apparent to one skilled in the art from the following description and claims when read in light of the accompanying drawings.

### BRIEF DESCRIPTION OF THE DRAWINGS

The present invention will be better understood by reading the Detailed Description of the Preferred and Alternate Embodiments with reference to the accompanying drawing figures, in which like reference numerals denote similar structure and refer to like elements throughout, and in which:

FIG. 1 is a front view of the identification card of an apparatus, method and system for the recordation, retrieval and management of pet information and capitalization of same according to a preferred embodiment of the present invention;

FIG. 2 is a rear view of the identification card of an apparatus, method and system for the recordation, retrieval and management of pet information and capitalization of same according to a preferred embodiment of the present invention;

FIG. 3 is flow diagram of the method and system of an apparatus, method and system for the recordation, retrieval and management of pet medical and health related information according to a preferred embodiment of the present invention;

FIG. 3A is flow diagram of the method and system of an apparatus, method and system for the recordation, retrieval and management of pet medical and health related information according to an alternate embodiment of the present invention; and,

FIG. 4 is flow diagram of the method and system of an apparatus, method and system for the recordation, retrieval and management of pet information and capitalization of same according to a preferred embodiment of the present invention.

### DETAILED DESCRIPTION OF THE PREFERRED AND ALTERNATIVE EMBODIMENTS

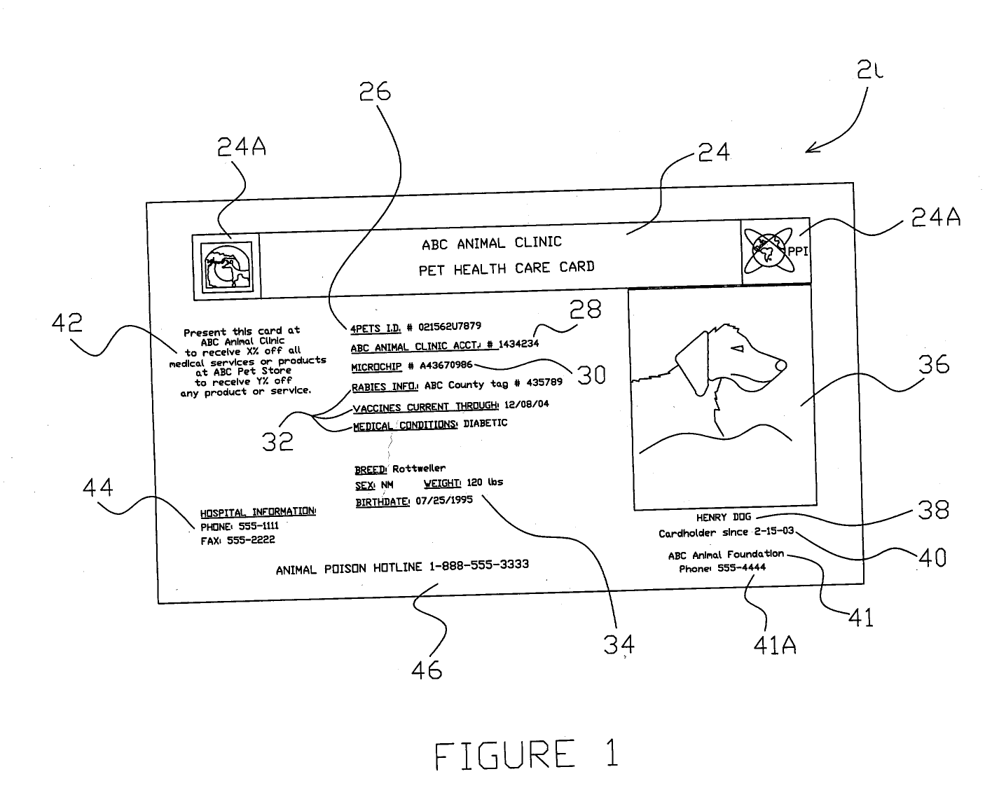

In describing the preferred and alternate embodiments of the present invention, as illustrated in FIGS. 1-4, specific terminology is employed for the sake of clarity. The invention, however, is not intended to be limited to the specific terminology so selected, and it is to be understood that each specific element includes all technical equivalents that operate in a similar manner to accomplish similar functions.

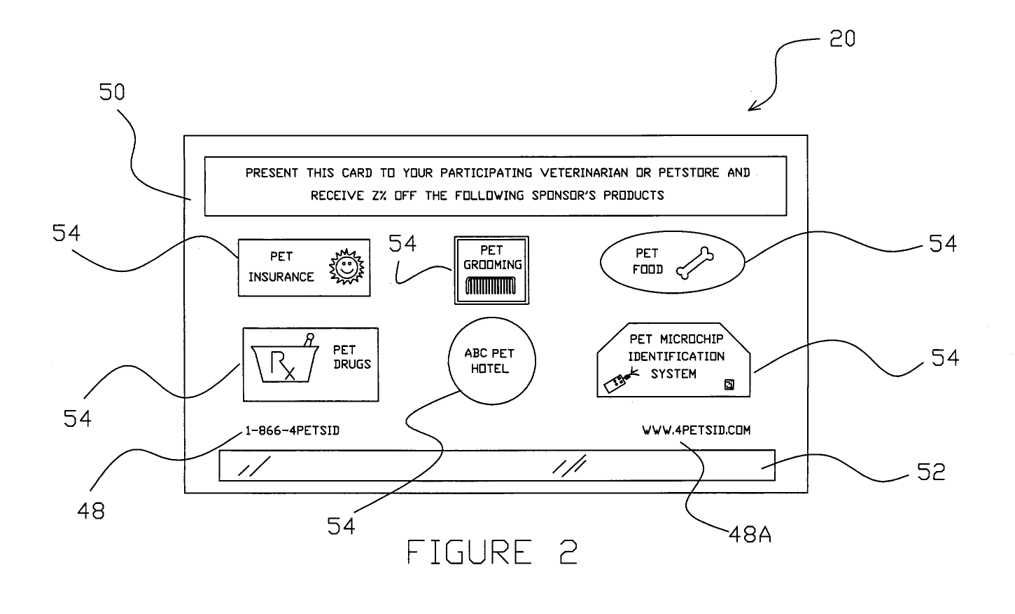

Referring now to FIGS. 1-2, the present invention in a preferred embodiment is an apparatus, method and system 10 for the recordation, retrieval and management of pet information and capitalization of same, preferably having, pet identification card 20 for implementation of the present method and system. Identification card 20 is preferably substantially dimensionally equivalent to conventional credit cards, and is preferably formed from a durable plastic or similar material; although, other dimensions and materials could be utilized. Preferably, card 20 possesses front side 22 and rear side 50 for the conveyance of relevant information thereon, wherein card 20 is preferably utilized by pet or animal owners (i.e., cardholders or consumers) to effectuate the recordation, retrieval and management of pertinent pet or animal information, and for the recordation and tracking of consumer demographics, demands, needs and spending habits for purposes more fully described below.

Preferably, and as more fully described below, promotional information (e.g., advertisements or brochures) about pet identification card 20 and associated membership programs is vendor-supplied to animal hospitals or other institutions for subsequent promotion and sale of same to pet or animal owners. Upon payment of the requisite fees by a pet owner, the vendor (i.e., preferably operating under the trademarks PPI and/or 4PETSID) preferably distributes card 20 and related membership program information to the pet owner (i.e., cardholder), as more fully described below. However, prior to vendor-distribution of card 20 and associated membership program information to the pet owner, the vendor must first acquire the relevant pet health information to be displayed on front side 22 of card 20 for each specific pet or animal. As such, the vendor preferably acquires the relevant pet health information from the location through which the pet owner purchased card 20 and the related membership program, wherein such a location is typically the animal hospital at which the pet is most often treated or examined, as such a location is more likely to possess the most thorough compilation of the pet's medical history. Preferably, release of such information is governed by pet owner authorization, or other legal measures, contractual or otherwise. Preferably, the relevant pet health and medical information acquired by the original card vendor is recorded, stored and managed by the vendor for subsequent retrieval and distribution of same for purposes more fully developed below.

It is contemplated in an alternate embodiment that the original card vendor could acquire relevant pet health information for display on front side 22 of card 20 from any other suitable source possessing or having access to such information, such as, for exemplary purposes only, humane societies, animal shelters, pet stores and/or obedience schools. Additionally, it is contemplated in yet another alternate embodiment that card 20 could be sold with blank data fields for insertion of relevant information therein, wherein such data or information would then be conveyed back to the original card vendor for recordation, storage and management of same, and the assignment of a membership number to the pet owner/card holder, as more fully described below.

Preferably, front side 22 possesses pet or animal organization name 24 and organization logo 24A, wherein organization name 24 and organization logo 24A are preferably representative of the animal hospital or other organization through which card 20 and associated membership programs will be sold, and/or the location in which the pet or animal is most frequently brought, and therefore the location which would possess the most complete compilation of the pet's health information and medical history. It is contemplated in an alternate embodiment, however, that the original card vendor could provide card 20 and related membership programs to other organizations, such as, for exemplary purposes only, pet stores, pet or animal organizations, obedience schools, humane societies, veterinarian hospitals, pet food and drug companies, pet insurance companies, and/or the like, for subsequent promotion and sale to pet owners, wherein the organization's name would then be represented by organization name 24 and corresponding organization logo 24A on front side 22 of card 20. As stated earlier, organization name 24 preferably represents the organization or entity that sells card 20 to the pet owner, wherein a portion of the sales price is preferably retained by the organization and the remaining portion thereof, transferred to the original card vendor, as more fully described below.

Front side 22 of card 20 further preferably possesses pet identification number 26 which represents or functions as the membership number of the pet and/or cardholder belonging to a selected membership program, as more fully addressed below; organization identification number 28, wherein organization identification number 28 preferably represents or corresponds to the patient or file number and account of the pet as categorized by the organization represented by organization name 24; and, microchip number 30, representative of the, microchip subcutaneously implanted in the pet for purposes of electronically tracking and identifying lost pets for reuniting same with the pet owner, wherein such microchip identification technology and services are known within the art and offered via organizations such as, for exemplary purposes only, HOMEAGAIN. It is contemplated in an alternate embodiment that card 20 could also possess relevant health insurance information and an associated identification number for in-depth access to same.

Front side 22 of card 20 further preferably possesses health or medical information 32, wherein medical information 32 preferably conveys information regarding rabies vaccinations and identification number, vaccination due dates, health examination due dates, current medical conditions such as, for exemplary purposes only, diabetes, kidney failure, heart failure, arthritis, blindness, hearing loss, drug allergies and/or any other health or medical related conditions or information, and wherein the current medical condition is preferably depicted in bold red print for purposes of saliency.

In addition to medical information 32, front side 22′ further preferably possesses biodata 34, wherein biodata 34 preferably conveys information regarding the pet's breed, sex, weight, birth date and/or other relevant information such as, for exemplary purposes only, eye color, dominant hair color, or the like. Furthermore, for purposes of visual identification, front side 22 of card 20 preferably possesses picture 36 of the pet or animal. Pet name 38 and cardholder date 40 are also preferably conveyed on front surface 22. Animal foundation name 41 and contact information 41A are preferably disposed on front side 22 of card 20, proximal cardholder date 40, wherein animal foundation name 41 and contact information 41A preferably enable a cardholder to utilize such information for purposes of making monetary donations, providing support, or the like, to the designated animal foundation. It is contemplated in an alternate embodiment that a picture of the pet owner could also be depicted on card 20 to facilitate accurate identification of the true pet owner should the pet depicted by picture 36 become lost and subsequently found, as more fully described below.

Front side 22 of card 20 further preferably possesses discount information 42, wherein discount information 42 preferably conveys information regarding the percentage of discount on veterinary or medical services, food and drug products, or the like, wherein use or presentation of card 20 during the purchase of such discounted products or services preferably records the purchase on card 20 and remotely, as more fully described below.

Front card 20 further preferably possesses telephone and facsimile numbers 44 of the organization represented by organization name 24, and further possesses animal poison hotline number 46. It is contemplated, however, that card 20 could possess any telephone, facsimile, or contact number desired, such as, for exemplary purposes only, local animal hospital telephone and facsimile numbers, pet owner telephone and facsimile numbers, insurance company telephone and facsimile numbers, humane society telephone and facsimile numbers, and/or the telephone and facsimile numbers of the original card vendor. Additionally, it is contemplated that card 20 could further possess the address information of the organization represented by organization name 24, or alternatively, the address information of local animal hospitals, the pet owner, insurance company, local humane society, original card vendor, and/or the like.

Preferably, all information conveyed on front side 22 is recorded, managed and at the access of the original card vendor for implementation of the present system and method, as more fully described below.

Rear side 50 of card 20 preferably possesses toll free number 48 and web address 48A for use by a cardholder, wherein calling toll free number 48, or accessing the website represented by web address 48A, and presenting identification number 26 preferably permits the cardholder to receive answers to questions and/or update relevant information via assistance from the original card vendor, or the like. Preferably, toll free number 48 and/or web address 48A and the services provided thereunder are accessible through certain membership programs of the present inventive system and method, as more fully described below. Rear side 50 further preferably possesses data strip 52, wherein data strip 52 is preferably any suitable input/output recordation, storage and retrieval means, such as, for exemplary purposes only, bar code technology, magnetic strips, and/or microchip technology. Data strip 52 preferably functions to record, store and permit access to a variety of information, such as, for exemplary purposes only, medical or health records, drug prescriptions, pet owner information, veterinarian fees and corresponding veterinarian services. Data strip 52 further preferably functions to record and track consumer spending habits and expenses attributed to the general maintenance and care of the pet or animal, as more fully described below. Additionally, any and all information on card 20 is preferably capable of being updated, wherein such updates may occur should there be any changes in health or medical conditions, or in any other relevant information.

Information presented on front surface 22 of card 20 and/or stored via data strip 52 is preferably utilized for a plurality of purposes. For instance, such information is preferably utilized by the pet owner and/or veterinarian to obtain relevant information therefrom for expeditious implementation of appropriate medical procedures, reminders, examinations and treatments (i.e., for expeditious first response medical action). Furthermore, because all the information presented on front surface 22 is preferably conveyed to the original card vendor for establishing a database of information for each cardholder, should a cardholder's pet or animal become lost, the original card vendor (i.e., PPI) can, via a database of veterinary hospitals, animal shelters, pet stores or the like, effectuate a mass mailing, facsimile, E-mailing, or the like, that would contain all the information on front side 22 of card 20, or a selection therefrom, for use by the finder of the lost pet, thus facilitating medical treatment of same and a more promising and expeditious reunion between the pet and pet owner. Additionally, should the cardholder misplace or lose his/her card 20, the cardholder may request issuance of another card 20 from the original vendor or the organization represented by organization name 24, preferably upon payment of the requisite fee. Alternatively, veterinary hospitals, animal shelters, pet stores or the like, could be provided with a digital camera, or the like, for taking pictures of the lost animals brought to them, wherein the digital images/pictures could then be conveyed to vendor 100, or the like, for comparison with pictures of pets or animals in vendor's 100 database for purposes of identifying the animal, and reuniting the animal with the pet or animal owner.

Information presented on front surface 22 of card 20 and/or stored via data strip 52 is further preferably utilized for animal boarding purposes. Specifically, when the pet or animal is being boarded at a boarding facility (i.e., animal shelter, animal hospital, animal hotel, animal humane society, and/or the like), information from card 20 may be utilized to determine if vaccinations are current prior to boarding the animal. Additionally, if the animal suffers from diabetes, arthritis or other medical conditions requiring regular monitoring, appropriate measures may be taken prior to boarding the animal to facilitate care of same.

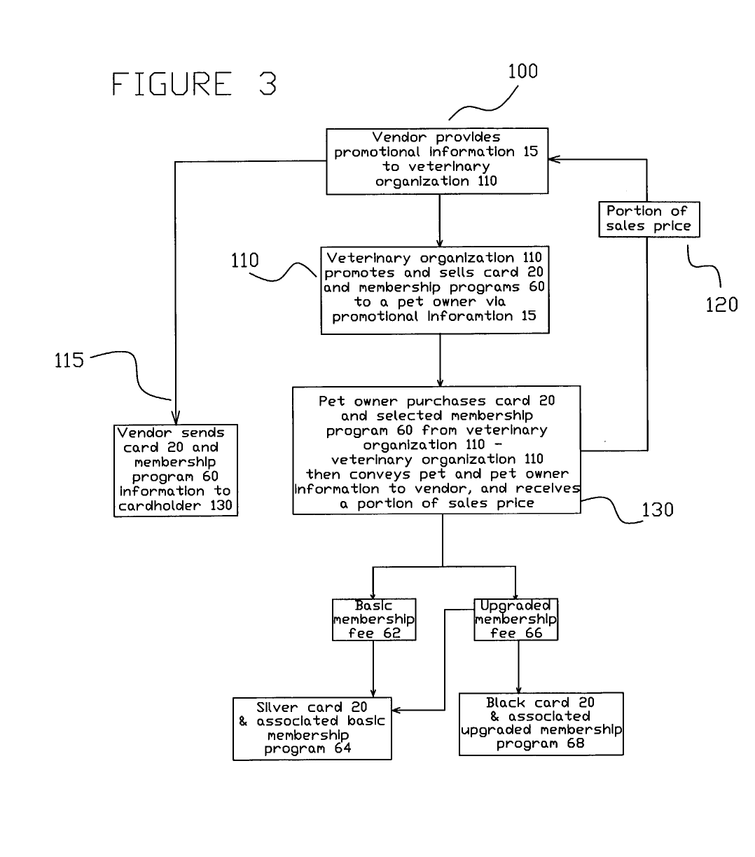

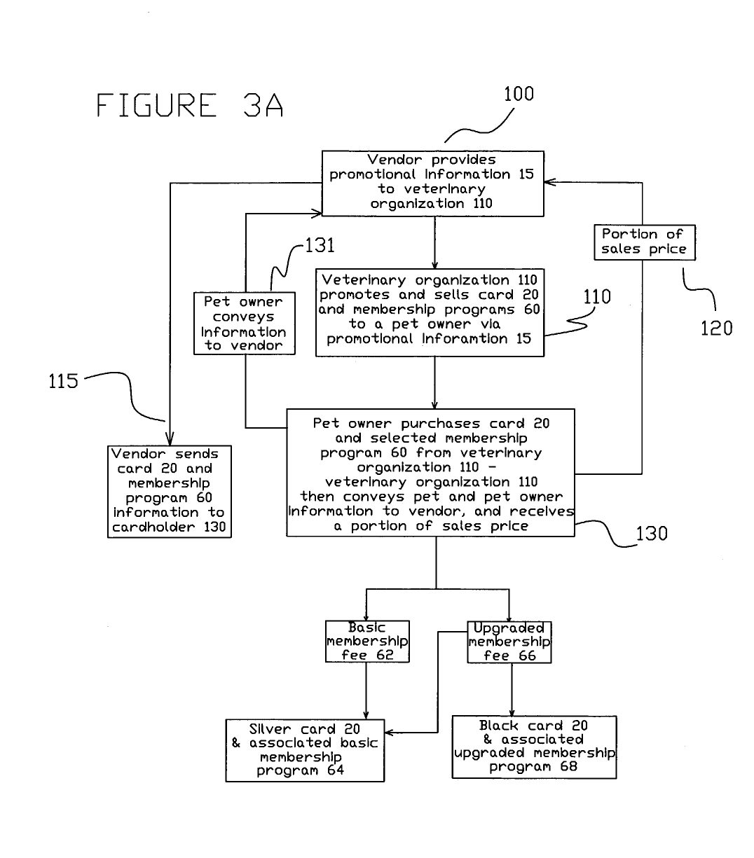

Referring now to FIG. 3, illustrated therein is a flow diagram of the present preferred method and system for the recordation, retrieval and management of pet medical and health related information as implemented via cardholder use of card 20 and associated membership programs. Specifically, vendor 100 provides veterinary organization 110, or the like, with promotional information 15 (e.g., brochures or advertisements) of card 20 and associated membership programs 60, wherein veterinary organization 110 preferably promotes and sells card 20 and an associated membership program 60 to a pet owner, and wherein vendor 100 and veterinary organization 110 each receive a portion 120 of the paid card 20 and membership fee for the specific type of membership program purchased. It is contemplated in an alternate embodiment, however, that veterinary organization 110 could receive any predetermined portion of the sales price of card 20 either individually, or in combination with a predetermined portion of the membership fee, wherein the portion of the membership could be an amount not necessarily correlated with the type of membership program purchased. Preferably, veterinary organization 110 coveys all relevant pet and pet owner information to vendor 100 for database development and records keeping; however, cardholder 130 could also convey 131 all relevant pet and pet owner information to vendor 100 (see FIG. 3A). Upon receiving all relevant information and related fees, vendor 100 preferably sends 115 card 20 to cardholder 130. Thereafter pet owner/cardholder 130 preferably utilizes card 20 and associated membership program 60 to his/her benefit. It is contemplated in an alternate embodiment that if cardholders 130 were to pay for card 20 and membership fee via credit card, the received payment or funds could be entirely transferred or deposited into vendor's 100 business account, wherein a designated portion of the funds would then subsequently be distributed to the appropriate veterinary organization 110 on a monthly basis, or other specified period of time.

Preferably, cardholder 130 may select from two different membership programs 60, wherein both membership programs 60 preferably offer a variety of goods and services; although, it is recognized that any number of membership programs could be established with any combination or selection of goods and services.

Preferably, upon a pet owner's payment of a minimum or basic membership fee 62 to veterinary organization 110, and following receipt of all relevant information and fees by vendor 100, and receipt of card 20 by cardholder 130, cardholder 130 is entitled to the benefits of membership program 64 (i.e., preferably marketed or advertised as the silver card or membership program). Preferably membership program 64 provides pet identification number 26, organization number 28, microchip number 30, medical information 32, biodata 34, picture 36, pet name 38, cardholder date 40, discount information 42 (e.g., X% off all medical services and/or products, Y% off any product and/or service from participating pet stores and/or veterinary hospital, Z% off any of the card's 20 sponsors products, etc.), telephone and facsimile numbers of organization 24 (i.e., veterinarian hospital, or the like), animal poison control hotline number 46, toll free number 48 and website address 48A and the associated services of answering questions and/or updating relevant information, or the like.

Preferably, upon a pet owner's payment of an upgraded membership fee 66 to veterinary organization 110, and following receipt of all relevant information and fees by vendor 100, and receipt of card 20 by cardholder 130, cardholder 130 is entitled to the benefits of membership program 68 (i.e., preferably marketed or advertised as the black card or membership program). Preferably membership program 68 provides all the benefits of membership program 64 in addition to providing cardholder 130 with a medical link or access to information on his/her pet's condition, along with recommendations on follow-up care at the office of veterinarian organization 110, and website access for reminders on vaccines, heartworm preventatives, blood work, health exams, and/or card 20 renewal reminders. It is contemplated that such reminders may be sent via an email, wherein the sender's screen name would be the name of the pet or animal; alternatively, vendor 100 and/or veterinarian organization 110 could also send such reminders. Additionally, through membership program 68, cardholder 130 will receive a quarterly news letter from vendor 100, or any other contracted party, wherein the newsletter preferably possesses general health information about pets/animals, pictures and a discussion or writing on the history and treatment of a pet voted as “pet of quarter,” and a website link or global networking system-based “chat room” link to other pet owners in membership program 68 that have pets or animals with the same or similar medical conditions for holding discussions with such pet owners.

Although the present method and system utilizes a membership program 60 having a two-tiered hierarchal structure, it is contemplated in an alternate embodiment that the present system and method could possess any number of membership levels. Additionally, it is contemplated that membership programs 64 and 68 could be selectively renewed following completion of a designated time period (i.e., quarterly, biannual or yearly renewals).

The present invention further provides a method and system for the retrieval and management of such consumer spending habit information, wherein a hierarchically-based sponsorship program preferably governs a particular sponsor's type and depth of access and/or retrieval of pet or animal owner spending habits and expenses attributed to the general maintenance and care of the pet or animal, thus permitting the sponsor to conduct market analyses of consumer demographics, demands, needs and spending habits for subsequent capitalization upon same via direct marketing, promotional discount offers, or the like.

Specifically, card 20 provides a variety of promotional offers and discounts on certain products and services, thereby encouraging cardholders to present their cards 20 upon purchasing preferably specific pet food and specific drug products, paying for specific veterinarian services, or the like. In addition to the promotional and discount offers provided via discount information 42 on front side 22 of card 20, rear side 50 preferably possesses sponsor logos 54 that preferably represent animal/pet food and drug manufacturers, distributors and/or retailers, insurance providers, pet tracking and identification services, obedience school services, and/or other providers of pet/animal products or services providers, such as, for exemplary purposes only, NOVARTIS, PFIZER, EUKANUBA, SCIENCE DIET, VETERINARY PET INSURANCE and/or HOMEAGAIN. As such, upon the cardholder's presentation of card 20 during the purchase of products and/or services from the sponsors represented by sponsor logos, the cardholder preferably receives a discount on his/her purchase.

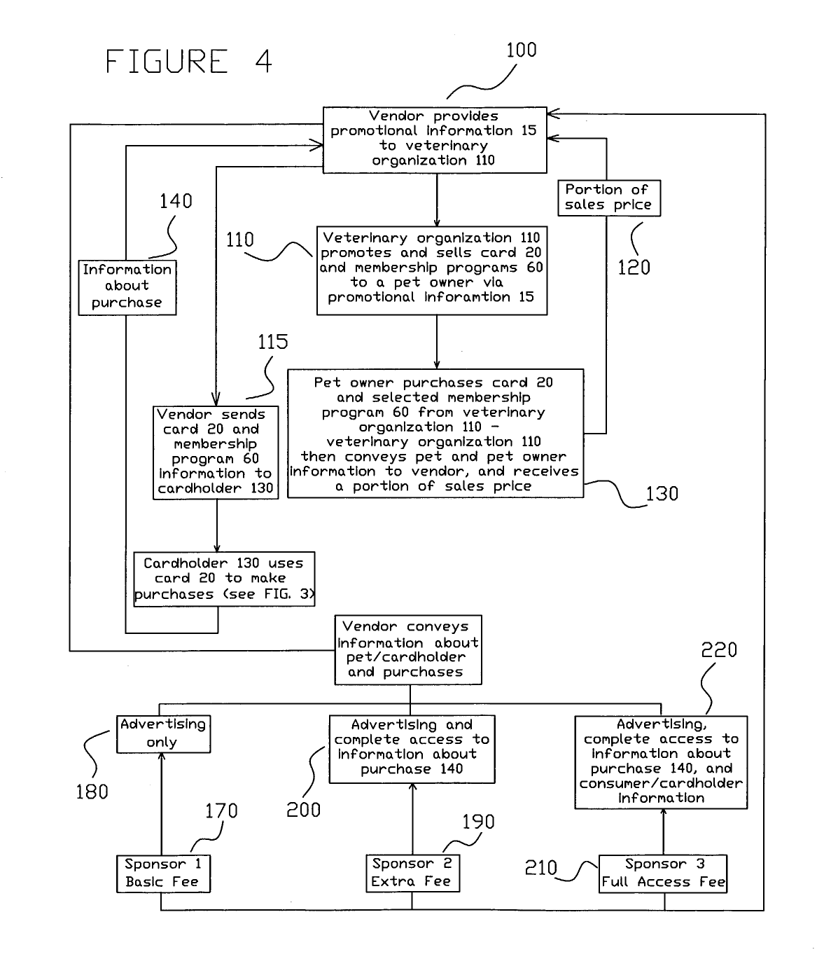

Referring now to FIG. 4, illustrated therein is a flow diagram of the present preferred method and system for-the recordation, retrieval and management of pet information and capitalization of same as implemented via cardholder use of card 20. Specifically, and as discussed above, vendor 100 provides veterinary organization 110, or the like, with promotional information 15 (e.g., brochures or advertisements) of card 20 and associated membership programs 60, wherein veterinary organization 110 preferably promotes and sells card 20 and an associated membership program 60 to a pet owner for the above-referenced applicable membership fee, and wherein vendor 100 and veterinary organization 110 each receive a portion 120 of the designated fee. As stated earlier, all relevant pet and pet owner information is conveyed to vendor 100 for database development and records keeping. Upon receiving all relevant information and related fees, vendor 100 preferably sends 115 card 20 to cardholder 130. Thereafter pet owner/cardholder 130 preferably utilizes card 20 to receive discounts on purchases of products and/or services as offered through discount information 42 and/or the sponsors designated on rear side 50 of card 20 by sponsor names 54.

Preferably each time card 20 is presented by cardholder 130 during the purchase of the aforementioned discounted products and/or services, card 20 is preferably scanned via the data strip 52 and thereby records and tracks the purchased discounted products or services. As such, card 20 records and tracks consumer spending habits and expenses attributed to the general maintenance and care of the pet or animal, wherein such information 140 is then conveyed to original card vendor 100 for recordation, management and subsequent distribution 150 via sponsorship program 160 to sponsors such as animal food and drug providers, insurance carriers and/or other pet related product or service vendors, as more fully detailed below. Alternatively, card 20 could record and/or track consumer spending habits and expenses on specific products and/or services.

Information 140 preferably enables original card vendor 100, the sponsors of card 20 represented by sponsor logos 54, and/or other organizations and entities, to analyze consumer/card holder 130 spending habits for determination of consumer geographic location, species of pet, desirable or undesirable product lines, profitable retail locations, profitable product or service lines, frequency of purchases, typical fees associated with veterinary services, effective food and drug products, or the like, wherein such market determinations and analyses are selectively accessible by sponsors of the present inventive system and method for subsequent capitalization upon same via direct consumer marketing, promotional discount offers, or the like.

Specifically, sponsor logos 54 are placed upon rear side 50 of card 20 upon payment by the corresponding sponsors of a minimum or basic sponsorship fee 170 to original card vendor 100, wherein basic sponsorship fee 170 preferably entitles the sponsor to basic advertising rights 180 by way of sponsor logo 54 on card 20. Should a sponsor desire to access and retrieve all the aforementioned consumer spending habit information 140 in addition to having basic advertising rights 180 on card 20, the sponsor preferably pays an extra fee 190 to original card vendor 100 for an advertising and complete information package 200. Additionally, the sponsor may elect to pay original card vendor 100 a full access fee 210 that would entitle the sponsor to access mailing, emailing and/or other similar contact information 220 of a specific consumer or cardholder for the direct marketing mailing, promotional and discount offerings thereto, wherein contact information 220 is preferably provided in addition to advertising and complete information package 200. Although the present method and system utilizes a sponsorship program preferably having a three-tiered hierarchal structure, it is contemplated in an alternate embodiment that the present system and method could possess any number of access levels, restrictions, and the like to information 150 and associated advertising services. Additionally, it is contemplated that the present sponsorship program could be selectively renewed following completion of a designated time period (i.e., monthly, quarterly, biannual or yearly renewals).

As stated above, analysis of information 140 preferably enables original card vendor 100, the sponsors of card 20 represented by sponsor logos 54, and/or other organizations and entities, to market to specific needs in the pet population and associated pet or animal owner demands, wherein such targeted markets may include owners of aged pets, pets with arthritis, pets with metabolic diseases, and/or any other medical conditions, or dietary demands.

Additionally, the database of information 140 held by vendor 100 is preferably adapted to automatically update cardholders 130, via E-mail or the like, of upcoming deadlines regarding required vaccinations, heartworm preventatives, blood work, ultrasounds, ECG's, X-rays, health exams, updated medical information, dental work, and/or reminders to owners of overweight pets that they must return to their veterinarian for weighing of the pet and for determination of diet change if needed.

Furthermore, as pet owners possess much pride in their pets or animals, it is contemplated that picture 36 of card 20 could be utilized to show the pet owner's friends and family a picture of his/her pet, wherein the presentation of card 20 would essentially effectuate another form of advertising for vendor 100, veterinarian organization 110 and the sponsors represented by sponsor logos 54 on card 20.

Moreover, in view of increased pet theft and the illegal sale of same to unbeknownst research facilities and/or humane societies purchasing the pet under the false pretext of “lost” or “abandoned” pets, vendor 100 may also provide research facilities, humane societies, or the like, with access to vendor's 100 database of pet and pet owner information for purposes of permitting such facilities to research the ownership of the pet, and reunite same with the true pet owner if possible. Research facilities and/or humane societies are given incentive to research the ownership of such pets or animals via vendor's 100 granting of a reward (i.e., money, monetary or equipment grants) for each pet reunited with the legal owner. Alternatively, such facilities could be provided with a digital camera, or the like, for taking pictures of the animals brought to them, wherein the digital images/pictures could then be conveyed to vendor 100, or the like, for comparison with pictures of pets or animals in vendor's 100 database for purposes of identifying the animal, and verifying or researching the ownership of the pet.

Law enforcement officials may also have access to vendor's 100 database for purposes of reuniting lost or retrieved pets with the legal owners of same.

It is contemplated in an alternate embodiment that card 20 could be in the form of a collar to be worn by the pet or animal, wherein the collar style card 20 would also possess data strip 52, thereby facilitating the expeditious provision and retrieval of pet health and identification related information for medical treatment and/or for effectuating a mass mailing or facsimile of the pet's information should the pet become lost and subsequently found by a non-owner.

It is contemplated in an alternate embodiment that card 20 could be in the form of a collar tag to be worn by the pet or animal, wherein the collar tag would also possess data strip 52, thereby facilitating the expeditious provision and retrieval of pet health and identification related information for medical treatment and/or for effectuating a mass mailing or facsimile of the pet's information should the pet become lost and subsequently found by a non-owner.

It is contemplated in an alternate embodiment that card 20 could be in the form of a Braille card for use by blind pet owners, especially those having seeing-eye dogs.

It is contemplated in an alternate embodiment that card 20 could be a credit card or debit card as well, thus further facilitating the recordation, retrieval and management process of consumer spending information.

It is contemplated in an alternate embodiment that credit card companies could license or buy the database of pet owner information and pet information so that each time a pet owner utilizes a particular credit card, the representative or associated credit card company would pay vendor 100 a royalty fee or the like.

It is contemplated in an alternate embodiment that the present invention could require sponsors to pay vendor 100 for each card 20 distributed and/or sold.

It is contemplated in an alternate embodiment that cards 20 could only be distributed/sold within a specific geographic region or segmented market.

It is contemplated in an alternate embodiment that the present invention could permit sponsors to request that the cards 20 possessing their logos 54 thereon be distributed/sold within a specific geographic region or segmented market.

It is contemplated in an alternate embodiment that the present invention could permit sponsors to engage in exclusive agreements with vendor 100 that would permit a particular sponsor to advertise its specific product or service line via particular logos, and that the cards 20 possessing such exclusive advertising rights/logos be distributed/sold within a specific geographic region or segmented market.

It is contemplated in an alternate embodiment that card 20 could possess the pet owner's address thereon for purposes of mailing lost cards 20 back to the appropriate cardholders 130.

It is contemplated in an alternate embodiment that veterinary organizations 110 could take photographs of all visiting patients, and retain such photographs on record until such time that the pet owner is ready to partake in a membership program 60. It is further contemplated that veterinary organizations 110 could utilize veterinarian hospital management software programs for purposes of maintaining a database of photographs for future use in a membership program 60, wherein such software programs, such as AVIMARK, are presently utilized for veterinarian hospital business management purposes and for managing digital photographic images of pet/animal patients, wherein such digital photographic images are taken by the veterinarian for animal identification purposes and for printing personalized purchase receipts, or the like.

It is contemplated in an alternate embodiment that card 20 could possess the requisite technology for downloading and/or uploading any desired information, wherein such technology would be in the form of a “smart-card” or similar device. It is also contemplated that any known technology may be utilized to convey energy or data such as, for exemplary purposes only, radio based tagging transponder systems, RFID technology, EAS technology and magnetic coupled techniques.

It is contemplated in an alternate embodiment that card 20 could be utilized by the pet owner to record future pet examination dates, future inoculation dates, amounts and types of current and future required prescription drugs, dietary regimens, or the like, wherein such information could be stored and retrieved by the pet owner/cardholder via a home scanning and information retrieval means.

It is contemplated in an alternate embodiment that card 20 could be in the form of a CD-ROM, minidisk, data stick, and/or other form of digital recordation and retrieval technology.

It is contemplated in an alternate embodiment that the present invention could be utilized for any type of animal, such as, for exemplary purposes only, wild animals for tagging and tracking health and behavior of same, farm animals and/or zoo animals.

It is contemplated in an alternate embodiment that the present invention could be utilized by adoption agencies or humane societies as a source of revenue, wherein distribution or sale of card 20 and associated membership programs 60 by adoption agencies or humane societies would effectively function as another avenue of advertisement for vendor 100, veterinary organization 110, sponsors, or the like. Specifically, adoption agencies or humane societies could provide an animal adopter with a temporary card 20 and membership program 60 upon payment for same, wherein a portion of the payment would be retained by the agency, and the remainder thereof, transferred to a veterinary organization having an exclusive relationship with that particular agency. As such, use of card 20 at the veterinary organization would entitle the cardholder to discounts and the like, and would further effectuate a form of advertisement for the veterinary organization, sponsors and vendor 100 for each card 20 sold by the agency. Moreover, should the cardholder renew his/her card 20 after completion of the temporary period, the agency may receive a portion of the renewal fee. Alternatively, the agency could have such exclusive relationships with a plurality of veterinary organizations, systematically selling a card 20 representative of each veterinary organization and retaining a portion of the sales price and/or renewal fee thereof; thus, each veterinary organization that the agency has a relationship with would have an equal opportunity to advertise their services, as would the sponsors and vendor 100. It is contemplated that the agency could receive revenue from the sponsors of card 20 for each card that is sold, instead of, or in addition to, the direct revenue associated with the sale and/or renewal of card 20 and membership programs 60.

It is contemplated in an alternate embodiment that veterinary organization 110, sponsors, or the like, could provide pet owners with a temporary card 20 and membership program 60 upon payment for same. Such temporary cards 20 would function to provide the pet owner with immediate access to discounts and related card 20 and membership program 60 benefits, as opposed to delaying such benefits until receipt of the hardcopy or permanent card 20. The temporary card 20 could come in the form of a “tear-out” card from a brochure, wherein the pet owner would fill the brochure out with all applicable pet and pet owner information, and wherein the brochure with the supplied information would then be conveyed to vendor 100 (i.e., PPI).

It is contemplated in an alternate embodiment that information retrieved in the present invention could be utilized for clinical or medical studies to determine the different types of veterinary treatments offered for different types of animal ailments, and thus, potentially establish a standard within professional societies for treatment of a particular type of medical treatment.

Having thus described exemplary embodiments of the present invention, it should be noted by those skilled in the art that the within disclosures are exemplary only, and that various other alternatives, adaptations, and modifications may be made within the scope of the present invention. Accordingly, the present invention is not limited to the specific embodiments illustrated herein, but is limited only by the following claims.

What is claimed is:

1. A system for storing, retrieving and managing animal information, comprising:
means for identifying the animal; and,
means for recording information related to the animal for subsequent management and retrieval of at least a portion of said information for at least one user-selected application.

2. The system of claim 1, wherein said identifying means is at least one identification card having animal identification information thereon, and wherein said animal identification information is selected from the group consisting of animal health, animal medical history, animal medical conditions, animal vaccination information, animal rabies identification number, animal drug allergies, animal physical appearance, animal owner information, picture of animal, picture of animal owner, animal hospital information, animal patient information, animal electronic identification information and animal microchip identification information.

3. The system of claim 1, wherein said at least one identification card further comprises discount offers on animal related goods and services, and wherein said animal related goods and services is selected from the group consisting of animal drug products, animal food products, animal care products, animal adoption agencies, animal hotel services, animal caretaker services, animal obedience school services, animal insurance, animal medical services and animal poison control information.

4. The system of claim 2, wherein said recording means is at least one recording medium carried by said at least one identification card, and wherein said at least one recording medium records current and updated said animal identification information.

5. The system of claim 4, wherein said at least one recording medium is selected from the group consisting of digital recording mechanisms, analog recording mechanisms, bar code technology, magnetic strip technology, microchip technology, radio frequency identification technology (RFID) and typed/written medium.

6. The system of claim 1, wherein said at least one user-selected application is selected from the group consisting of animal medical treatment, animal identification, lost animal recovery services and animal owner identification.

7. A system of recording, tracking and managing consumer spending habits related to the maintenance of an animal, comprising:
means for identifying the animal; and,
means for recording information related to consumer purchases of goods and services utilized to maintain the animal.

8. The system of claim 7, wherein said identifying means is at least one identification card having animal identification information thereon, and wherein said animal identification information is selected from the group consisting of animal health, animal medical history, animal medical conditions, animal vaccination information, animal drug allergies, animal physical appearance, animal owner information, picture of animal, picture of animal owner, animal hospital information, animal store information, animal patient information, animal electronic identification information, animal poison control information and animal microchip identification information.

9. The system of claim 7, wherein said at least one identification card further comprises discount offers on animal related goods and services, and wherein said animal related goods and services is selected from the group consisting of animal drug products, animal food products, animal care products, animal adoption agencies, animal insurance, lost animal recovery services and animal medical services.

10. The system of claim 8, wherein said recording means is at least one recording medium carried by said at least one identification card, and wherein said at least one recording medium records current and updated said animal identification information.

11. The system of claim 10, wherein said at least one recording medium comprises digital recording mechanisms, analog recording mechanisms, bar code technology, magnetic strip technology, microchip technology, radio frequency identification technology (RFID), typed/written medium, and/or similar technology.

12. The system of claim 7, wherein said information related to consumer purchases of goods and services is selected from the group consisting of product brand name, service brand name, product type, service type, discounted products, discounted services, animal drug products, animal food products, animal care products, animal medical services, animal health conditions, consumer geographic location, specie of animal, desirable product lines, undesirable product lines, profitable retail locations, frequency of consumer purchases, financial data, typical product fees and typical service fees.

13. A method of recording, tracking and managing consumer spending habits related to the maintenance of an animal, comprising the steps of:
a. providing a consumer with a means for recording purchases of goods and services utilized to maintain the animal, wherein the consumer can present said recording means during purchase of said goods and services; and
b. recording the purchase of said goods and services via said recording means.

14. The method of claim 13, wherein said recording means is carried by at least one identification card having animal identification information thereon, and wherein said animal identification information is selected from the group consisting of animal health, animal medical history, animal medical conditions, animal vaccination information, animal drug allergies, animal physical appearance, animal owner information, picture of animal, picture of animal owner, animal hospital information, animal patient information, animal electronic identification information, animal poison control information and animal microchip identification information.

15. The method of claim 14, wherein said at least one identification card further comprises discount offers on animal related goods and services, and wherein said animal related goods and services is selected from the group consisting of animal drug products, animal food products, animal care products, animal insurance, animal adoption services, lost animal recovery services and animal medical services.

16. The method of claim 14, wherein said recording means is at least one recording medium carried by said at least one identification card, and wherein said at least one recording medium records current and updated said animal identification information.

17. The method of claim 16, wherein said at least one recording medium is selected from the group consisting of digital recording mechanisms, analog recording mechanisms, bar code technology, magnetic strip technology, radio frequency identification technology (RFID) and microchip technology.

18. A system of advertising and direct marketing toward animal owners, comprising:
means for identifying the animal; and,
means for recording information related to animal owner purchases of goods and services utilized to maintain the animal, wherein at least a portion of recorded information is retrievable by at least one advertiser.

19. The system of claim 18, wherein said identifying means is at least one identification card having animal identification information thereon, and wherein said animal identification information is selected from the group consisting of animal health, animal medical history, animal medical conditions, animal vaccination information, animal drug allergies, animal physical appearance, animal owner information, animal hospital information, animal patient information, picture of animal, picture of owner, animal electronic identification information and animal microchip identification information.

20. The system of claim 19, wherein said at least one identification card further comprises discount offers on animal related goods and services, and wherein said animal related goods and services is selected from the group consisting of animal drug products, animal food products, animal care products, animal insurance, animal adoption services, lost animal recovery services and animal medical services.

21. The system of claim 19, wherein said recording means is at least one recording medium carried by said at least one identification card, and wherein said at least one recording medium records current and updated said animal identification information.

22. The system of claim 21, wherein said at least one recording medium is selected from the group consisting of digital recording mechanisms, analog recording mechanisms, bar code technology, magnetic strip technology, radio frequency identification technology (RFID) and microchip technology.

23. The system of claim 18, wherein said recorded information is selected from the group consisting of product brand name, service brand name, product type, service type, discounted products, discounted services, animal drug products, animal food products, animal care products, animal medical services, consumer geographic location, specie of animal, desirable product lines, undesirable product lines, profitable retail locations, financial data, frequency of consumer purchases, typical product fees and typical service fees.

24. The system of claim 23, wherein said at least a portion of said recorded information is retrievable by said at least one advertiser upon payment of a fee.

25. The system of claim 24, wherein said at least one advertiser utilizes said at least a portion of said recorded information to directly market and advertise to the animal owner.

26. The system of claim 24, wherein said at least one advertiser possesses the option of advertising its company logo on said identifying means.

27. A method of advertising and marketing, comprising the steps:
a. providing an animal owner with a means for recording information related to the purchase of goods and services utilized to maintain the animal, wherein said owner can present said recording means during purchase of said goods or services; and
b. recording said information related to the purchase of said goods and services via said recording means; and,
c. permitting advertisers to access at least a portion of said information.

28. The method of claim 27, wherein said recording means is carried by at least one identification card having animal identification information thereon, and wherein said animal identification information is selected from the group consisting of animal health, animal medical history, animal medical conditions, animal vaccination information, animal drug allergies, animal physical appearance, animal owner information, picture of animal, picture of animal owner, animal hospital information, animal patient information, animal electronic identification information and animal microchip identification information.

29. The method of claim 28, wherein said at least one identification card further comprises discount offers on animal related goods and services, and wherein said animal related goods and services is selected from the group consisting of animal drug products, animal food products, animal care products, animal insurance, animal adoption services, lost animal recovery services and animal medical services.

30. The method of claim 28, wherein said recording means is at least one recording medium carried by said at least one identification card, and wherein said at least one recording medium records current and updated said animal identification information.

31. The method of claim 30, wherein said at least one recording medium is selected from the group consisting of digital recording mechanisms, analog recording mechanisms, bar code technology, magnetic strip technology, radio frequency identification technology (RFID), microchip technology and written/typed medium.

32. The method of claim 27, wherein said information is selected from the group consisting of product brand name, service brand name, product type, service type, discounted products, discounted services, animal drug products, animal food products, animal care products, animal medical services, consumer geographic location, species of animal, desirable product lines, undesirable product lines, profitable retail locations, financial data, frequency of consumer purchases, typical product fees and typical service fees.

33. The method of claim 32, wherein said at least a portion of said information is retrievable by said advertisers upon payment of a fee.

34. The method of claim 27, wherein said advertisers utilize said at least a portion of said information to directly market and advertise to said animal owner.

35. The system of claim 34, wherein said advertisers possesses the option of advertising their company logo on said identifying means.

36. A system of advertising and direct marketing toward animal owners, comprising:
means for identifying the animal;
means for recording information related to animal owner purchases of goods and services utilized to maintain the animal; and
means for subscribing to at least a portion of said information recorded via said recording means, wherein an animal owner, a veterinarian, an advertiser or a credit company may access at least a portion of said recorded information.

37. The system of claim 36, wherein said identifying means is at least one identification card having animal identification information thereon, and wherein said animal identification information is selected from the group consisting of animal health, animal medical history, animal medical conditions, animal vaccination information, animal drug allergies, animal physical appearance, animal owner information, picture of animal, picture of animal owner, animal hospital information, animal patient information, animal electronic identification information and animal microchip identification information.

38. The system of claim 37, wherein said at least one identification card further comprises discount offers on animal related goods and services, and wherein said animal related goods and services is selected from the group consisting of animal drug products, animal food products, animal care products, animal insurance, animal adoption agencies, lost animal recovery services and animal medical services.

39. The system of claim 36, wherein said recording means is at least one recording medium carried by said at least one identification card, and wherein said at least one recording medium records current and updated said animal identification information.

40. The system of claim 39, wherein said at least one recording medium is selected from the group consisting of digital recording mechanisms, analog recording mechanisms, bar code technology, magnetic strip technology, radio frequency identification technology (RFID) and microchip technology.

41. The system of claim 36, wherein said information is selected from the group consisting of product brand name, service brand name, product type, service type, discounted products, discounted services, animal drug products, animal food products, animal care products, animal medical services, consumer geographic location, specie of animal, desirable product lines, undesirable product lines, profitable retail locations, frequency of consumer purchases, purchase patterns, financial data, typical product fees and typical service fees.

42. The system of claim 36, wherein said means for subscribing to said at least a portion of said information is a sponsorship program that governs a sponsor's type and depth of access to said information.

43. The system of claim 42, wherein payment of at least one fee by said sponsor governs said sponsor's type and depth of access to said at least a portion of said information.

44. The system of claim 43, wherein said sponsor, at owner's consent, utilizes said at least a portion of said information to directly market and advertise to said animal owner.

45. The system of claim 44, wherein said sponsor possesses the option of advertising said sponsor's company logo on said identifying means.

46. A method of advertising and direct marketing toward animal owners, comprising the steps:
a. providing an animal owner with a means for recording information related to the purchase of goods and services utilized to maintain the animal;
b. presenting said recording means during purchase of said goods and services;
c. recording said information related to the purchase of said goods and services via said recording means; and,
d. subscribing for access to at least a portion of said information recorded via said recording means.

47. The method of claim 46, wherein said recording means is carried by at least one identification card having animal identification information thereon, and wherein said animal identification information is selected from the group consisting of animal health, animal medical history, animal medical conditions, animal vaccination information, animal rabies identification information, animal drug allergies, animal physical appearance, animal owner information, animal hospital information, animal patient information, picture of animal, picture of animal owner, animal electronic identification information and animal microchip identification information.

48. The method of claim 47, wherein said at least one identification card further comprises discount offers on animal related goods and services, and wherein said animal related goods and services is selected from the group consisting of animal drug products, animal food products, animal care products, animal insurance, animal adoption services, lost animal recovery services, animal medical services and animal poison control information.

49. The method of claim 46, wherein said recording means is at least one recording medium carried by said at least one identification card, and wherein said at least one recording medium records current and updated said animal identification information.

50. The method of claim 49, wherein said at least one recording medium is selected from the group consisting of digital recording mechanisms, analog recording mechanisms, bar code technology, magnetic strip technology, radio-based transponder and microchip technology.

51. The method of claim 46, wherein said information is selected from the group consisting of product brand name, service brand name, product type, service type, discounted products, discounted services, animal drug products, animal food products, animal care products, animal medical services, consumer geographic location, species of animal, animal insurance data, desirable product lines, undesirable product lines, profitable retail locations, frequency of consumer purchases, financial data, typical product fees and typical service fees.

52. The method of claim 46, wherein said step of subscribing for access to said at least a portion of said information is through a sponsorship program that governs a sponsor's type and depth of access to said information.

53. The method of claim 52, further comprising the step of requiring said sponsor to pay at least one fee that governs said sponsor's type and depth of access to said at least a portion of said information.

54. The method of claim 53, further comprising the step of permitting said sponsor, with owner's consent, to utilize said at least a portion of said information to directly market and advertise to said animal owner.

55. The method of claim 54, further comprising the step of presenting to said sponsor the option of advertising said sponsor's company logo on said identifying means.

56. A system of advertising and direct marketing toward animal owners, comprising:
means for identifying the animal;
means for recording information related to the health and medical conditions of the animal; and,
means for subscribing to at least a portion of said information recorded via said recording means.

57. The system of claim 56, wherein said identifying means is at least one identification card having animal identification information thereon, and wherein said animal identification information is selected from the group consisting of animal health, animal medical history, animal medical conditions, animal vaccination information, animal drug allergies, animal physical appearance, animal owner information, animal hospital information, animal patient information, picture of animal, picture of animal owner, animal electronic identification information- and animal microchip identification information.

58. The system of claim 57, wherein said at least one identification card further comprises discount offers on animal related goods and services, and wherein said animal related goods and services is selected from the group consisting of animal drug products, animal food products, animal care products, animal insurance, animal adoption services, lost animal recovery services and animal medical services.

59. The system of claim 56, wherein said recording means is at least one recording medium carried by said at least one identification card, and wherein said at least one recording medium records current and updated said animal identification information.

60. The system of claim 59, wherein said at least one recording medium is selected from the group consisting of digital recording mechanisms, analog recording mechanisms, bar code technology, magnetic strip technology, radio frequency identification technology (RFID) and microchip technology.

61. The system of claim 56, wherein said information is selected from the group consisting of animal health, animal medical history, animal medical conditions, animal vaccination information, animal drug allergies, animal hospital information and animal patient information.

62. The system of claim 56, wherein said means for subscribing to said at least a portion of said information is a sponsorship program that governs a medical industry sponsor's type and depth of access to said information.

63. The system of claim 62, wherein payment of at least one fee by said medical industry sponsor governs said medical industry sponsor's type and depth of access to said at least a portion of said information.

64. The system of claim 63, wherein said medical industry sponsor utilizes said at least a portion of said information to directly market and advertise medically related goods and services, and coupons and incentives to the animal owner for care of the animal.

65. The system of claim 64, wherein said medical industry sponsor possesses the option of advertising said medical industry sponsor's company logo on said identifying means.

66. A method of advertising and direct marketing toward animal owners, comprising the steps:
a. providing an animal owner with a means for recording information related to the health and medical conditions of the animal;
b. presenting said recording means during medical examinations of the animal;
c. recording said information related to the health and medical conditions of the animal via said recording means; and,
d. subscribing for access to at least a portion of said information recorded via said recording means.

67. The method of claim 66, wherein said recording means is carried by at least one identification card having animal identification information thereon, and wherein said animal identification information is selected from the group consisting of animal health, animal medical history, animal medical conditions, animal vaccination information, animal drug allergies, animal physical appearance, animal owner information, picture of animal, picture of animal owner, animal hospital information, poison control information, animal patient information, animal electronic identification information and animal microchip identification information.

68. The method of claim 67, wherein said at least one identification card further comprises discount offers on animal related goods and services, and wherein said animal related goods and services is selected from the group consisting of animal drug products, animal food products, animal care products, animal insurance, animal adoption services, lost animal recovery services and animal medical services.

69. The method of claim 66, wherein said recording means is at least one recording medium carried by said at least one identification card, and wherein said at least one recording medium records current and updated said animal identification information.

70. The method of claim 69, wherein said at least one recording medium is selected from the group consisting of digital recording mechanisms, analog recording mechanisms, bar code technology, magnetic strip technology, radio frequency identification technology (RFID) and microchip technology.

71. The method of claim 66, wherein said information is selected from the group consisting of animal health, animal medical history, animal medical conditions, animal vaccination information, animal drug allergies, animal hospital information and animal patient information.

72. The method of claim 66, wherein said step of subscribing for access to said at least a portion of said information is through a sponsorship program that governs a medical industry sponsor's type and depth of access to said information.

73. The method of claim 72, further comprising the step of requiring said medical industry sponsor to pay at least one fee that governs said medical industry sponsor's type and depth of access to said at least a portion of said information.

74. The method of claim 73, further comprising the step of permitting said medical industry sponsor to utilize said at least a portion of said information to directly market and advertise to said animal owner for care of the animal.

75. The method of claim 74, further comprising the step of presenting to said medical industry sponsor the option of advertising said medical industry sponsor's company logo on said identifying means.

76. The method of claim 66, further comprising the step of providing selected owner information to at least one credit provider, wherein said credit provider issues credit to a portion of said owners, and wherein said credit provider shares a portion of charges made with said credit.

77. A system of locating and identifying lost pets, comprising:
means for identifying the animal;
means for recording information related to the animal; and,
means for effectuating mass distribution of recorded information related to the animal to facilitate the process of locating and identifying the lost animal.

78. The system of claim 77, wherein said identifying means is at least one identification card having animal identification information thereon, and wherein said animal identification information is selected from the group consisting of animal health, animal medical history, animal medical conditions, animal vaccination information, animal drug allergies, animal physical appearance, animal owner information, animal hospital information, animal patient information, picture of animal, picture of animal owner, poison control information, animal electronic identification information and animal microchip identification information.

79. The system of claim 78, wherein said at least one identification card further comprises discount offers on animal related goods and services, and wherein said animal related goods and services is selected from the group consisting of animal drug products, animal food products, animal care products, animal insurance, animal adoption services, lost animal recovery services and animal medical services.

80. The system of claim 77, wherein said recording means is at least one recording medium carried by said at least one identification card, and wherein said at least one recording medium records current and updated said animal identification information.

81. The system of claim 80, wherein said at least one recording medium is selected from the group consisting of digital recording mechanisms, analog recording mechanisms, bar code technology, magnetic strip technology, radio frequency identification technology (RFID) and microchip technology.

82. The system of claim 77, wherein said means for effectuating mass distribution of recorded information related to the animal is a mass facsimile of at least a portion of said recorded information to a plurality of locations, said plurality of location selected from the group consisting of veterinary hospitals, animal shelters, research facilities, humane societies, general retail stores and pet stores.

83. The system of claim 77, wherein said means for effectuating mass distribution of recorded information related to the animal is a mass mailing of at least a portion of said recorded information to a plurality of locations, said plurality of location selected from the group consisting of veterinary hospitals, animal shelters, general retail stores and pet stores.

84. The system of claim 77, wherein said means for effectuating mass distribution of recorded information related to the animal is a mass E-mailing of at least a portion of said recorded information to a plurality of locations, said plurality of location selected from the group consisting of veterinary hospitals, animal shelters, general retail stores and pet stores.
